(19) United States
(12) Patent Application Publication
(10) Pub. No.: US 2005/0234417 A1
(43) Pub. Date: Oct. 20, 2005

(54) Sanitary napkin

(75) Inventors: Wataru Yoshimasa, Kagawa (JP); Yuuki Noda, Kagawa (JP); Megumi Tokumoto, Mitoyo-gun (JP); Noritatsu Tamagawa, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-shi (JP)

(21) Appl. No.: 11/097,494

(22) Filed: Apr. 1, 2005

(30) Foreign Application Priority Data
Apr. 15, 2004 (JP) 2004-119854

(51) Int. Cl. A61F 13/15; A61F 13/20

(52) U.S. Cl. 604/378; 604/358

(57) ABSTRACT

Disclosed is a sanitary napkin which is allowed to quickly absorb menstrual blood and also effective in preventing absorbed menstrual blood from returning to the skin-side surface under pressure and in concealing the color of absorbed menstrual blood from the outside. Between a liquid absorbent layer and a topsheet formed of a resin film having a large number of liquid passage apertures, there is disposed a barrier layer formed of a resin film having a large number of liquid passage apertures. The barrier layer is composed of a plurality of strip-shaped elements, and liquid passage regions are defined between adjacent elements of the barrier layer. After passing through the liquid passage apertures of the topsheet, menstrual blood tends to be absorbed by the liquid absorbent layer through the liquid passage regions. The barrier layer functions to prevent liquid return and conceal the color of menstrual blood.

### BACKGROUND OF THE INVENTION

1. Field of the Invention

The present invention relates to a sanitary napkin which is effective in preventing lateral leakage and concealing the color of menstrual blood absorbed in a liquid absorbent layer. More particularly, the invention relates to a sanitary napkin which is intended to diffuse an applied liquid longitudinally beneath a topsheet for quick absorption into the liquid absorbent layer.

2. Description of the Related Art

Sanitary napkins are typically constructed to include a liquid-permeable topsheet, a backsheet, and a liquid absorbent layer (or absorbent core) disposed between the topsheet and the backsheet. The sanitary napkins need to be able to absorb menstrual blood in a short period of time and to be effective in preventing lateral leakage of an applied liquid and also in preventing menstrual blood absorbed in the liquid absorbent layer from returning to the skin-side surface of the topsheet, and it is also desired that the color of menstrual blood absorbed in the liquid absorbent layer is less noticeable from the outside. To satisfy these requirements, there have been developed topsheets of various structures and additional components to be disposed between the topsheet and the liquid absorbent layer.

Patent Publication 1 identified below discloses a sanitary napkin which is intended to facilitate intake of liquid, to be effective in preventing liquid return to the skin-side surface of the topsheet, and to improve the function of concealing the color of liquid. In the sanitary napkin disclosed in Patent Publication 1, more specifically, a first passage layer (topsheet) located on its skin-side surface is a resin film formed with large openings having an individual open area of 1.4 to 3.0 mm² and a second passage layer located beneath the first passage layer is formed of highly-porous lofty elastic fibrous material. Also disclosed is a structure to be compared with the invention, where the same first passage layer is employed but the second passage layer is replaced by a resin film formed with small openings.

On the other hand, absorbent articles in which a second layer is disposed between the liquid-permeable topsheet and the liquid absorbent layer are disclosed in Patent Publications 1, 2 and 3 identified below. The second layer is a nonwoven fabric formed with a large number of recesses toward the liquid absorbent layer. When a large amount of liquid is applied in a short period of time, the recesses function to hold liquid within to improve the effect of preventing lateral leakage. In addition, liquid applied to the recesses is diffused inside the second layer to suppress liquid return to the skin-side surface of the topsheet.
Patent Publication 1: Japanese Unexamined Patent Publication No. H11-507573
Patent Publication 2: Japanese Unexamined Patent Publication No. 2000-140015
Patent Publication 3: Japanese Unexamined Patent Publication No. S55-094251
Patent Publication 4: Japanese Unexamined Patent Publication No. H06-038998

In the invention disclosed in Patent Publication 1, liquid-permeability of the first passage layer is improved by the large openings, and the second passage layer formed of the fibrous material is disposed between the first passage layer and the liquid absorbent layer. When body pressure is exerted on the skin-side surface, therefore, menstrual blood absorbed in the liquid absorbent layer is liable to pass through the second passage layer to return to the skin-side surface of the first passage layer through the large openings. In addition, since menstrual blood is allowed to be retained in the voids of the second passage layer, the color of menstrual blood is noticeable from the outside through the large openings of the first passage layer.

In the comparative structure disclosed in Patent Publication 1, on the other hand, the resin film having small openings is provided as the second passage layer beneath the first passage layer. This second passage layer appears to be able to make the color of menstrual blood inside the liquid absorbent layer less noticeable from the outside. However, the second passage layer also functions as a barrier against menstrual blood tending toward the liquid absorbent layer after passing through the first passage layer. Therefore, menstrual blood is liable to remain between the first passage layer and the second passage layer and this residual menstrual blood tends to diffuse along a plane between the first passage layer and the second passage layer. This easily leads to lateral leakage. In addition, the residual menstrual blood easily returns to the skin-side surface of the first passage layer through the large openings.

In the absorbent articles disclosed in Patent Publications 2 to 4, on the other hand, liquid after passing through the topsheet is supposed to be temporarily held in the recesses of the second layer of a nonwoven fabric and then absorbed by the liquid absorbent layer through the second layer. If these absorbent articles are employed for disposable diapers and a large amount of liquid with relatively low viscosity such as urine is applied in a short period of time, the second layer functions to hold urine temporarily in the recesses and to allow passage of urine through spaces between fibers for subsequent absorption by the liquid absorbent layer. If they are employed for sanitary napkins, however, since menstrual blood with relatively high viscosity cannot easily pass through the spaces between fibers of the second layer, menstrual blood tends to remain in the recesses and return to the skin-side surface of the liquid-permeable topsheet.

### SUMMARY OF THE INVENTION

The present invention has been developed to solve the problems in the prior art set forth above and has an object to provide a sanitary napkin which is allowed to quickly intake menstrual blood with relatively high viscosity and is effective in preventing lateral diffusion and concealing the color of menstrual blood.

According to the invention, there is provided a sanitary napkin comprising
a liquid-permeable topsheet intended to face the vaginal opening of a wearer,
a backsheet located on opposite side of the topsheet, and
a liquid absorbent layer located between the topsheet and the backsheet, the sanitary napkin further comprising:
a barrier layer having a plurality of elements and located between the topsheet and the liquid absorbent layer, the elements being spaced laterally and elongated longitudinally of the sanitary napkin, at least a skin-side surface of each element being made of resin, a plurality of longitudinally elongated liquid passage regions being defined between adjacent elements of the barrier layer to allow liquid passage therethrough and subsequent liquid absorption by the liquid absorbent layer.

In the sanitary napkin of the present invention, the barrier layer functions to prevent liquid return from the liquid absorbent layer to the topsheet and to conceal the color of menstrual blood absorbed in the liquid absorbent layer. Due to the longitudinally elongated liquid passage regions defined between adjacent elements of the barrier layer, moreover, menstrual blood after passing through the topsheet can be absorbed by the liquid absorbent layer while being diffused longitudinally. Accordingly, menstrual blood can be quickly given to the liquid absorbent layer. This also results in prevention of lateral diffusion of menstrual blood to improve the effect of preventing lateral leakage of menstrual blood.

Preferably, the individual elements of the barrier layer have a plurality of liquid passage apertures so that menstrual blood applied to the topsheet can be quickly transferred to the liquid absorbent layer. In this case, more preferably, the topsheet has a skin-side surface made of resin and a plurality of liquid passage apertures, wherein in an area where the topsheet overlaps with the barrier layer, the liquid passage apertures in the individual elements of the barrier layer have a smaller individual open area than the liquid passage apertures in the topsheet and the individual elements of the barrier layer have a smaller percent open area due to the liquid passage apertures than the topsheet. With the individual open area of the liquid passage apertures being set larger in the topsheet, liquid hardly remains on the skin-side surface of the topsheet. With the individual open area of the liquid passage apertures being set smaller in the individual elements of the barrier layer, liquid return from the liquid absorbent layer toward the topsheet is reduced, while the effect of concealing the color of menstrual blood absorbed in the liquid absorbent layer is improved.

In one preferred embodiment, the individual elements of the barrier layer are in contact with the topsheet and the liquid absorbent layer. In this case, the elements may be completely separated from each other or partially connected to each other at longitudinally spaced locations.

In another preferred embodiment, the barrier layer is of a two-layer structure where an upper barrier layer is located on a lower barrier layer. In this case, more preferably, the upper and lower barrier layers are located in such a staggered manner that the elements of the lower barrier layer are located beneath the liquid passage regions defined between adjacent elements of the upper barrier layer so as to be effective in preventing liquid return from the liquid absorbent layer to the topsheet.

In still another preferred embodiment, a corrugated sheet is located between the topsheet and the liquid absorbent layer to function as the barrier layer at top portions thereof. In this case, menstrual blood after passing through the topsheet flows down valleys of the corrugated sheet longitudinally of the sanitary napkin for quick absorption by the liquid absorbent layer. More preferably, the corrugated sheet is apertured for liquid passage. Particularly in the slopes, the corrugated sheet may have larger liquid passage apertures than the topsheet so that menstrual blood in the valleys can be quickly absorbed by the liquid absorbent layer.

### BRIEF DESCRIPTION OF THE DRAWINGS

FIG. 1 is a plan view of a sanitary napkin according to a first embodiment of the present invention;

FIG. 2 is a sectional view the sanitary napkin of FIG. 1 taken along line II-II;

FIG. 3 is a partial enlarged view of FIG. 2;

FIG. 4 is a partial perspective view schematically showing how a topsheet and a barrier layer are disposed on a liquid absorbent layer;

FIG. 5 is a close-up picture of the skin-side surface of the sanitary napkin of FIG. 1;

FIG. 6 is an enlarged sectional view of a sanitary napkin being a modification of the first embodiment;

FIG. 7 is an enlarged perspective view of a modification of the barrier layer;

FIG. 8 is an enlarged plan view of a modification of the barrier layer;

FIG. 9 is a partial sectional view of a modification of the barrier layer;

FIG. 10 is a partial sectional view of a modification of the barrier layer;

FIG. 11 is a partial enlarged view of a sanitary napkin according to a second embodiment of the present invention;

FIG. 12 is a partial perspective view of the sanitary napkin according to the second embodiment;

FIG. 13 is a partial perspective view of a sanitary napkin being a modification of the second embodiment;

FIGS. 14(A) and 14(B) are partial perspective views showing preferred structures of the apertured sheet according to the second embodiment;

FIG. 15 is a perspective view showing a modification of the apertured sheet according to the second embodiment;

FIG. 16 is a perspective view showing a modification of the apertured sheet according to the second embodiment;

FIG. 17 is a perspective view showing a modification of the apertured sheet according to the second embodiment; and

FIG. 18 is a plan view of a sanitary napkin having a different structure according to the present invention.

### DESCRIPTION OF THE PREFERRED EMBODIMENT

It should be noted that the sanitary napkin, as well as its individual components, has two major surfaces: of which one surface intended to be worn toward the wearer's crotch is referred to as “skin-side surface”, while the other surface is referred to as “garment-side surface”.

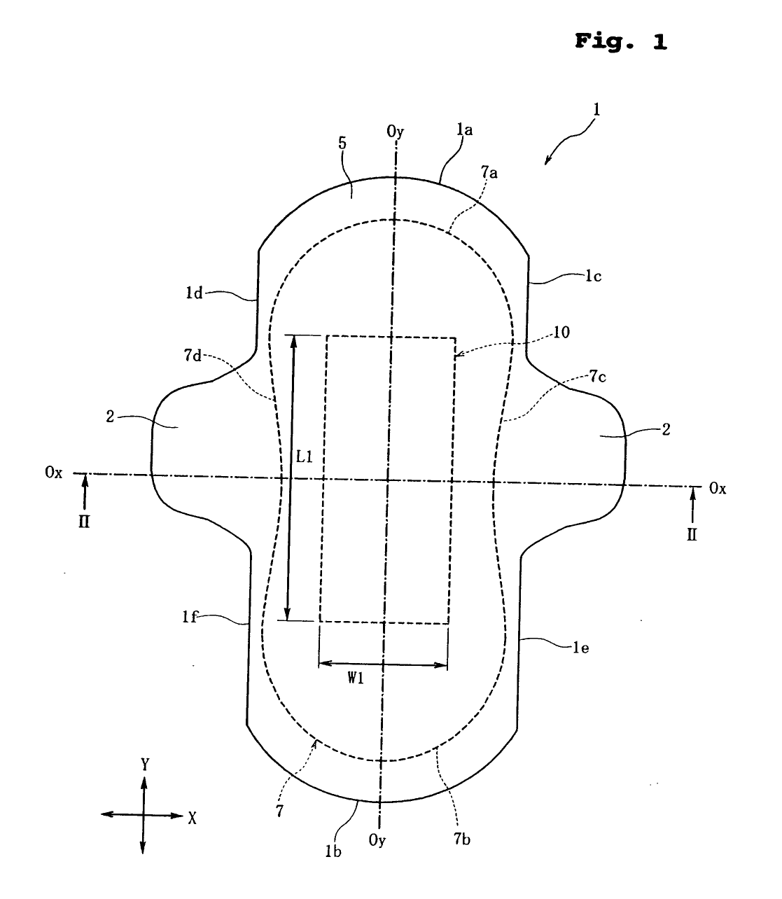

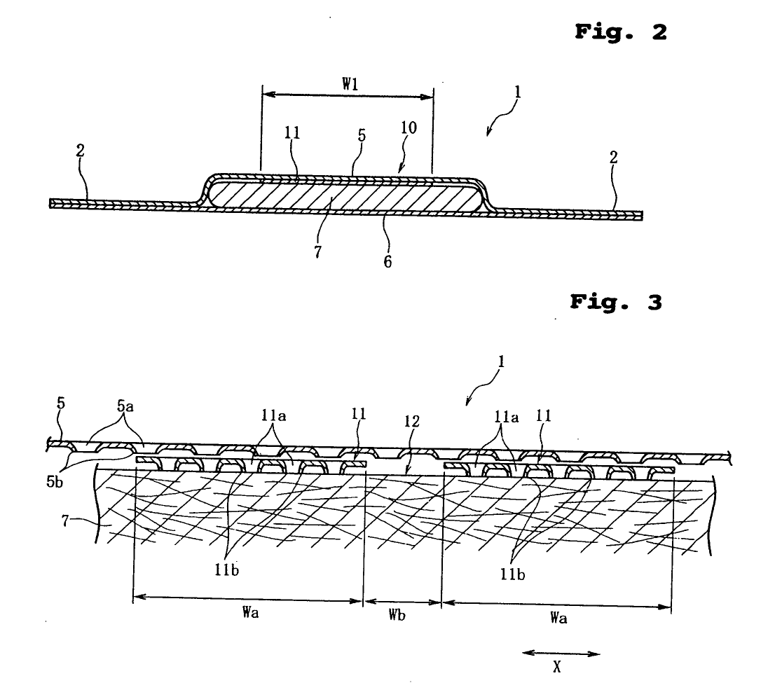

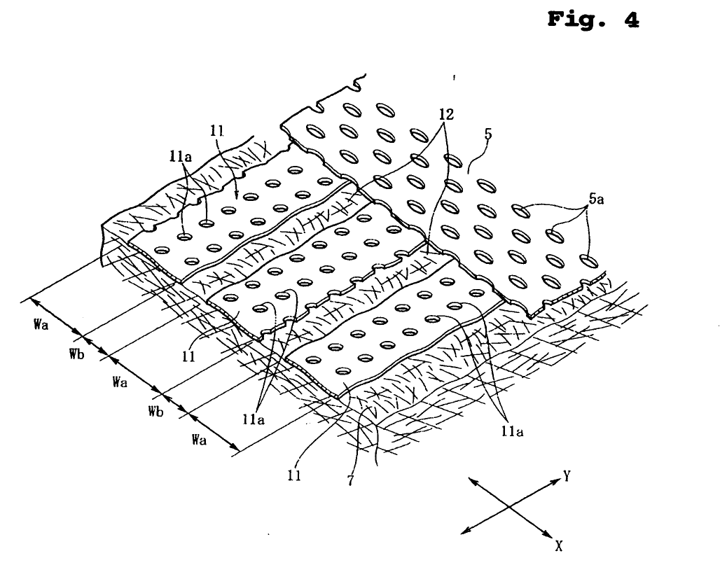

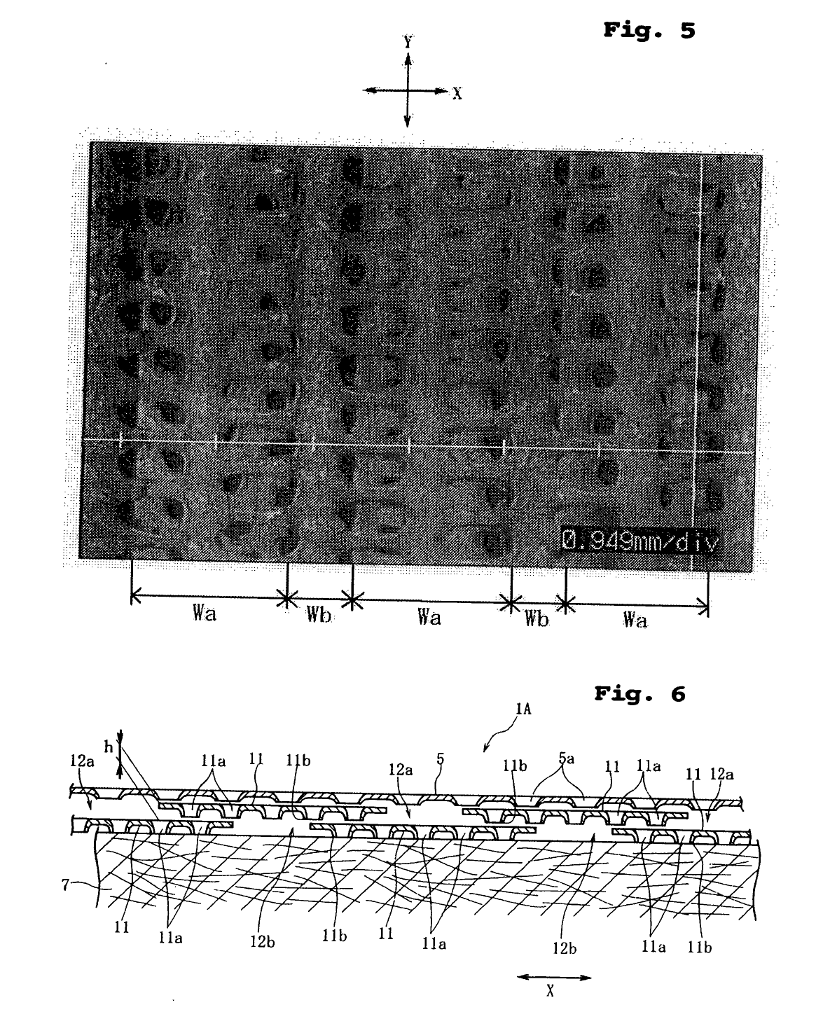

FIG. 1 is a plan view of a sanitary napkin 1 according to a first embodiment of the present invention with its skin-side surface directed to this side; FIG. 2 is a sectional view taken along line II-II of FIG. 1; FIG. 3 is a partial enlarged view of FIG. 2; FIG. 4 is a partial perspective view schematically showing how a topsheet and a barrier layer are disposed on a liquid absorbent layer; and FIG. 5 is a close-up picture of the skin-side surface of the sanitary napkin 1.

As shown in FIG. 1, the sanitary napkin 1 has a main body elongated in a Y-direction (or longitudinal direction) and bilaterally symmetrical about a longitudinal centerline Oy. The main body has front and rear edges 1a, 1b that are bowed (or bulged) forward and rearward, respectively. From laterally opposite sides of the main body, wings 2, 2 project outward. These wings 2, 2 are not aligned with a lateral centerline Ox and are located closer to the front edge 1a than to the rear edge 1b. The main body has forward right and forward left edges 1c, 1d forward of the wings 2, 2 and rearward right and rearward left edges 1e, 1f rearward of the wings 2, 2.

As shown in FIG. 2, the sanitary napkin 1 is basically constructed of a liquid-permeable topsheet 5 appearing on the skin-side surface of the sanitary napkin 1, a liquid-blocking backsheet 6 appearing on the garment-side surface of the sanitary napkin 1, and a liquid absorbent layer 7 disposed between the topsheet 5 and the backsheet 6. As indicated by dotted line in FIG. 1, the liquid absorbent layer 7 has a front edge 7a that is bowed (or bulged) forward and spaced inward from the front edge 1a of the sanitary napkin 1 and a rear edge 7b that is bowed (or bulged) rearward and spaced inward from the rear edge 1b of the sanitary napkin 1. The liquid absorbent layer 7 also has a right edge 7c that is spaced inward from the forward right edge 1c and the rearward right edge 1e of the sanitary napkin 1 and a left edge 7d that is spaced inward from the forward left edge 1d and the rearward left edge if of the sanitary napkin 1. Outside the liquid absorbent layer 7, the topsheet 5 and the backsheet 6 are bonded together through a hot-melt type adhesive.

If desired, additional liquid-impermeable side sheets may be located a given distance apart from the longitudinal centerline Oy to cover laterally opposite side portions of the skin-side surface of the sanitary napkin 1. The side sheets may also be used to provide leakage preventing walls (or cuffs) which extend longitudinally and are raised away from the skin-side surface.

In the sanitary napkin 1, the region enclosed by dotted line is designated main liquid-receiving region 10. The main liquid-receiving region 10 is symmetrical about both the longitudinal centerline Oy and the lateral centerline Ox and has a length L1 and a width W1, as shown in FIG. 1. In the main liquid-receiving region 10, a barrier layer 11 is disposed between the topsheet 5 and the liquid absorbent layer 7.

In the first embodiment, the barrier layer 11 comprises a plurality of strip-shaped elements (or strips) each having a width Wa in an X-direction (or lateral/transverse direction) of the sanitary napkin 1 and extending continuously over the length L1 of the main liquid-receiving region 10. The elements are made of resin film. FIG. 4 shows only three elements, but more than three elements are arranged at intervals in the X-direction to provide the main liquid-receiving region 10. The individual elements may be shorter than the length L1 of the main liquid-receiving region 10, if desired.

Liquid passage regions 12 are defined between adjacent elements. The individual liquid passage regions 12 have a width Wb in the X-direction and extend continuously over the length L1 of the main liquid-receiving region 10. In other words, the elements of the barrier layer 11 are spaced a distance Wb apart in the X-direction.

The length L1 and the width W1 of the main liquid-receiving region 10 are so set as to allow the main liquid-receiving region 10 to easily face the vaginal opening when the sanitary napkin 1 is worn in the crotch of a woman. The preferred range of the length L1 is (but not limited to) from 40 to 70 mm and the preferred range of the width W1 is (but not limited to) from 20 to 30 mm.

The width Wb of each liquid passage region 12 is, for example, from 0.5 to 10 mm, preferably from 1 to 8 mm, more preferably from 3 to 8 mm. The width Wb may be equal to or smaller or larger than the width Wa of each element of the barrier layer 11. For example, the total area of the liquid passage regions 12 is from 5 to 70% of the area (L1×W1) of the main liquid-receiving region 10. However, it is preferred that the width Wb is equal to or smaller than the width Wa and the total area of the liquid passage regions 12 is from 25 to 50% of the area (L1×W1) of the main liquid-receiving region 10. Within this range, menstrual blood absorbed in the liquid absorbent layer 7 can be effectively prevented from returning to the topsheet 5 due to the presence of the barrier layer 11 and the color of menstrual blood absorbed in the liquid absorbent layer 7 can also be effectively concealed by the barrier layer 11 and the topsheet 5. In addition, the liquid passage regions 12 facilitate absorption of menstrual blood into the liquid absorbent layer 7.

In the sanitary napkin 1 according to the first embodiment, the topsheet 5 is formed of an apertured synthetic resin film, i.e., a synthetic resin film in which a large number of liquid passage apertures 5a are regularly or randomly distributed. For the barrier layer 11, there is also used an apertured synthetic resin film. In the individual elements of the barrier layer 11, therefore, a large number of liquid passage apertures 11a are regularly or randomly distributed.

At least within the main liquid-receiving region 10: the individual open area of the liquid passage apertures 5a in the topsheet 5 is larger than (preferably 1.5 to 10 times, more preferably 2 to 6 times) that of the liquid passage apertures ha in the elements of the barrier layer 11; the percent open area of the topsheet 5 due to the liquid passage apertures 5a is larger than (preferably 1.5 to 10 times, more preferably 2 to 6 times) that of the elements of the barrier layer 11 due to the liquid passage apertures 11a. Here, the pitch of the liquid passage apertures 5a and the pitch of the liquid passage apertures 11a differ from each other in both the longitudinal direction and the lateral direction. FIG. 5 is a close-up picture of the main liquid-receiving region 10 actually fabricated according to the first embodiment. As viewed from above the skin-side surface, overlaps between the liquid passage apertures 5a and the liquid passage apertures 11a are appropriately distributed over the main liquid-receiving region 10.

For the topsheet 5 and the barrier layer 11, for example, there may be used the same apertured resin film. This resin film may be stretched to increase both the individual open area and the percent open area for the topsheet 5. In FIG. 5, more specifically, the resin film is stretched in the X-direction to have liquid passage apertures 5a elongated in the X-direction. For the barrier layer 11, on the other hand, the resin film may be used without stretching. In this case, the elements of the barrier layer 11 are of a larger thickness than the topsheet 5, and the liquid passage regions 12 defined between adjacent elements of the barrier layer 11 serve as channels having a depth equal to the thickness of the elements. The channels function to lead menstrual blood in the Y-direction for quick absorption of menstrual blood into the liquid absorbent layer 7. The channels also function to prevent leakage of menstrual blood in the X-direction.

The liquid passage apertures 11a have an open diameter of 0.3 to 3.5 mm (preferably 1.0 to 2.5 mm) and an individual open area of 0.07 to 9.61 mm² (preferably 0.78 to 4.91 mm²). The individual elements of the barrier layer 11 have a percent open area of 20 to 60% (preferably 35 to 50%) due to the liquid passage apertures 11a and a thickness of 0.15 to 1.0 mm (preferably 0.3 to 0.7 mm). For the topsheet 5, the same resin film as used for the barrier layer 11 is preferably stretched to 1.5 to 4 times its original length.

Such liquid passage apertures may be formed by a perforation method, for example, such that a resin film is softened on a substrate having a number of apertures and air is suck from the underside of the substrate. According to this perforation method, the individual liquid passage apertures 11a are tapered toward the liquid absorbent layer 7 to have rings (or short tubes) 11b projecting from the garment-side surface of the resin film, as shown in FIG. 3. Likewise, the individual liquid passage apertures 5a are tapered toward the liquid absorbent layer 7 to have rings (or short tubes) 5b projecting from the garment-side surface of the resin film.

Suitable thermoplastic resin materials for the topsheet 5 and the barrier layer 11 includes, but not limited to, LLDPE (linear low-density polyethylene), LDPE (low-density polyethylene), HDPE (high-density polyethylene), PP (polypropylene), TAFMER® by Mitsui Chemicals, INC., and blends thereof. These resin materials may also be combined to form a multi-layered film. The resin film may be whitened by kneading the resin material with inorganic filler such as titanium oxide, barium sulfate or calcium carbonate in an amount of 10 to 40 wt. %. If the topsheet 5 and the barrier layer 11 are whitened, the color of menstrual blood absorbed in the liquid absorbent layer 7 can be effectively concealed. Moreover, the resin material used for the topsheet 5 and the barrier layer 11 may be treated to be hydrophilic by adding a hydrophilizing agent such as esters in an amount of 0.1 to 2.5 wt. %.

The elements of the barrier layer 11 are fixed to the topsheet 5 such as by applying a hot-melt type adhesive in an amount of 1 to 10 g/m² so as not to interfere with liquid transfer or by heating them under pressure to have embossments in a dot pattern. In the latter case, the embossments occupy 1 to 40%, preferably 1 to 15% of the area (L1×W1) of the main liquid-receiving region 10.

The backsheet 6 is formed of a liquid-blocking sheet such as a PE (polyethylene) resin film. The liquid absorbent layer 7 is constructed by wrapping a mixture of fluff pulp and superabsorbent polymer (SAP) in tissue. So as not to interfere with liquid transfer, the elements of the barrier layer 11 are bonded to the liquid absorbent layer 7 by applying a hot-melt type adhesive in an amount of 1 to 10 g/m². The backsheet 6 is also bonded to the liquid absorbent layer 7 through a hot-melt type adhesive.

When wearing, the sanitary napkin 1 is placed on the inner side of a crotch part of an undergarment and fixed to the crotch part through a pressure-sensitive adhesive layer provided on the garment-side surface (backsheet 6) of the main body of the sanitary napkin 1. Then, the wings 2, 2 are folded back and adhered to the outer side of the crotch part of the undergarment through pressure-sensitive adhesive layers provided on the garment-side surface (backsheet 6) of the wings 2, 2.

When the sanitary napkin 1 is put on the crotch of a wearer, the main liquid-receiving region 10 faces the vaginal opening so that menstrual blood discharged from the vaginal opening can be applied mainly to the main liquid-receiving region 10. Menstrual blood applied to the main liquid-receiving region 10 passes through the relatively large liquid passage apertures 5a of the topsheet 5, comes into contact with the liquid absorbent layer 7 mainly in the liquid passage regions 12, and is then absorbed by the liquid absorbent layer 7 due to its hydrophilic power. Where the topsheet 5 overlaps with the elements of the barrier layer 11, as shown in FIG. 5, part of menstrual blood applied to the main liquid-receiving region 10 passes through the overlaps between the liquid passage apertures 5a and the liquid passage apertures 11a to reach the liquid absorbent layer 7.

Particularly when a large amount of menstrual blood is applied to the main liquid-receiving region 10 in a short period of time, menstrual blood after passing through the liquid passage apertures 5a is led along the liquid passage regions 12 in the Y-direction for quick absorption by the liquid absorbent layer 7. Here, lateral leakage of menstrual blood is inhibited by the relatively thick elements defining therebetween the liquid passage regions 12.

In the sanitary napkin 1, since the topsheet 5 has a relatively large percent open area and the liquid passage apertures 5a have a relatively large individual open area, an applied liquid hardly remains on the skin-side surface of the topsheet 5. In addition, since the topsheet 5 overlaps with the elements of the barrier layer 11 to decrease the area of the liquid absorbent layer 7 exposed externally through the liquid passage apertures, as shown in FIG. 5, menstrual blood absorbed in the liquid absorbent layer 7 is prevented from easily returning to the skin-side surface of the topsheet 5 and the color of menstrual blood absorbed in the liquid absorbent layer 7 becomes less noticeable from the outside.

FIG. 6 is a partial enlarged sectional view corresponding to FIG. 3 and showing a sanitary napkin 1A being a modification of the first embodiment. In the main liquid-receiving region 10 of the sanitary napkin 1A of FIG. 6, two barrier layers 11, 11 are stacked in a two-layer structure between the topsheet 5 and the liquid absorbent layer 7. The liquid passage regions defined between adjacent elements of the upper barrier layer 11 is designated 12a, while the liquid passage regions defined between adjacent elements of the lower barrier layer 11 is designated 12b. In both the upper and lower barrier layers 11, 11, the elements extend in the Y-direction and are spaced apart in the X-direction.

It should be noted that the upper and lower barrier layers 11, 11 are offset from one another in the X-direction such that the elements of the lower barrier layer 11 are located beneath the liquid passage regions 12a while the elements of the upper barrier layer 11 are located above the liquid passage regions 12b. As shown in FIG. 6, the elements of the barrier layers 11 have the liquid passage apertures 11a with the rings 11b projecting toward the liquid absorbent layer 7 due to the perforation method. Since at least the upper barrier layer 11 has the rings 11b, a clearance “h” being the total of a height of the ring 11b and a thickness of the resin film is provided between the topsheet 5 and the lower barrier layer 11.

The upper and lower barrier layers 11, 11 thus stacked are more effective in preventing menstrual blood absorbed in the liquid absorbent layer 7 from returning to the skin-side surface of the topsheet 5 and make the color of menstrual blood absorbed in the liquid absorbent layer 7 less noticeable from the outside. Even in this case, menstrual blood applied to the main liquid-receiving region 10 is allowed to quickly reach the liquid absorbent layer 7 due to the presence of the liquid passage regions 12a, 12b alternating with each other in the X-direction.

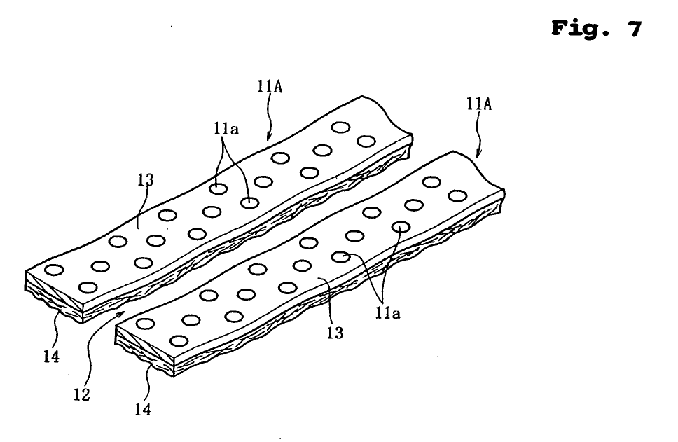

FIG. 7 is a partial enlarged perspective view of a barrier layer 11A being a modification of the barrier layer 11 employed in the foregoing embodiments. The barrier layer 11A comprises a plurality of elements each having a resin film layer 13 and a fibrous layer 14 joined to the resin film layer 13. The resin film layer 13 is whitened and treated to be hydrophilic in the same manner as the barrier layer 11 in the foregoing embodiments. The barrier layer 11A is also formed with the liquid passage apertures 11a. The liquid passage apertures 11a may pass through only the resin film layer 13 or both the resin film layer 13 and the fibrous layer 14. The fibrous layer 14 is a hydrophilic nonwoven fabric and joined to the resin film layer 13 such as by adhesive-bonding or fusion-bonding. For example, the fibrous layer 14 may be fusion-bonded to the resin film 13 to have embossments in a dot pattern. Suitable fibers for the fibrous layer 14 are synthetic resin fibers treated to be hydrophilic, such as PE, PP or PET (polyethylene terephthalate). The fibrous layer 14 may further contain cellulosic fibers.

In the embodiment of FIG. 7, since the hydrophilic fibrous layer 14 appears on the side edges of each element, menstrual blood after passing through the liquid passage apertures 5a of the topsheet 5 can be strongly drawn to the liquid passage regions 12 defined between adjacent elements of the barrier layer 11A.

FIG. 8 is an enlarged plan view of a barrier layer 11B being another modification. The barrier layer 11B comprises a plurality of elements each having a varying width. In the elements, more specifically, wide portions and narrow portions alternate with each other in the Y-direction. Also in liquid passage regions 12d defined between adjacent elements of the barrier layer 11B, accordingly, wide portions and narrow portions alternate with each other in the Y-direction.

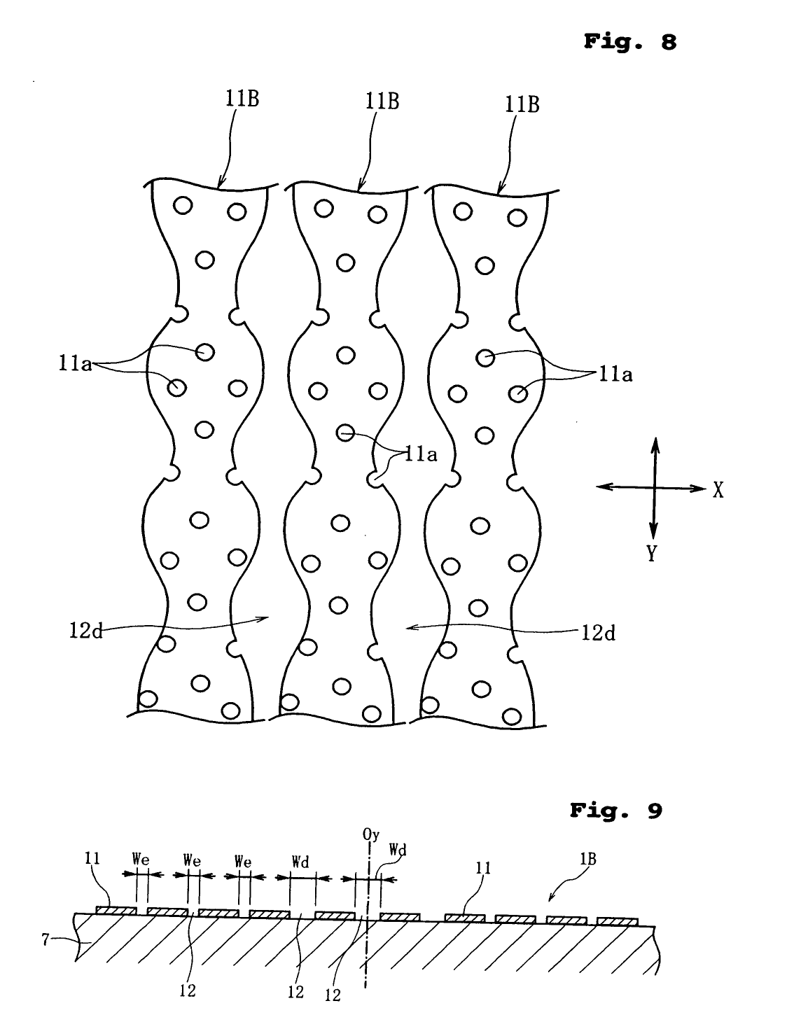

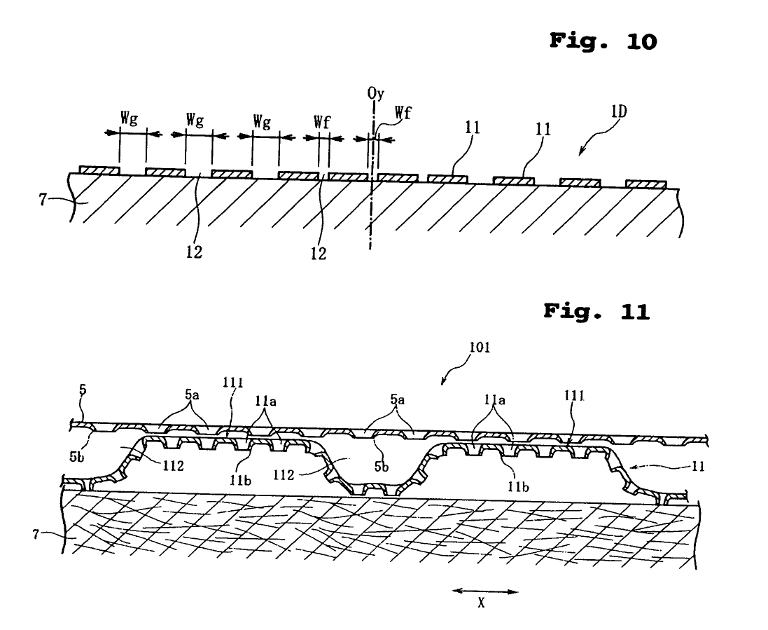

FIGS. 9 and 10 show sanitary napkins 1B and 1D being modifications of the foregoing embodiments, wherein the topsheet 5 is omitted from the drawings to clearly show the barrier layer 11 on the liquid absorbent layer 7.

In the sanitary napkin 1B of FIG. 9, the elements of the barrier layer 11 are spaced a relatively large distance Wd apart in a center region which extends along the longitudinal centerline Oy with a given width, while spaced a relatively small distance We apart outside the center region. In other words, the liquid passage regions 12 located in the center region have a width Wd which is larger than a width We of the liquid passage regions 12 located outside the center region. In this sanitary napkin 1B, menstrual blood after passing through the topsheet 5 can be led to the liquid absorbent layer 7 faster in the center region than in the rest of the main liquid-receiving region 10.

In the sanitary napkin 1D of FIG. 10, conversely, the liquid passage regions 12 located in the center region have a width Wf which is smaller than a width Wg of the liquid passage regions 12 located outside the center region.

The widths Wd, Wg may be in the range of more than 0.5 to 10 mm, preferably 1 to 5 mm. The widths We, Wf are smaller than the widths Wd, Wg but at least 0.5 mm.

Alternatively, the liquid passage regions 12 may have different widths which gradually increase or decrease depending on distances from the longitudinal centerline Oy in the X-direction.

Also in these sanitary napkins, the width of each element may be varied as in the barrier layer 11B.

In the foregoing embodiments, the elements are completely separated from each other to define a single liquid passage region therebetween, but the elements may be connected to each other to longitudinally divide each liquid passage region into two or more separate regions.

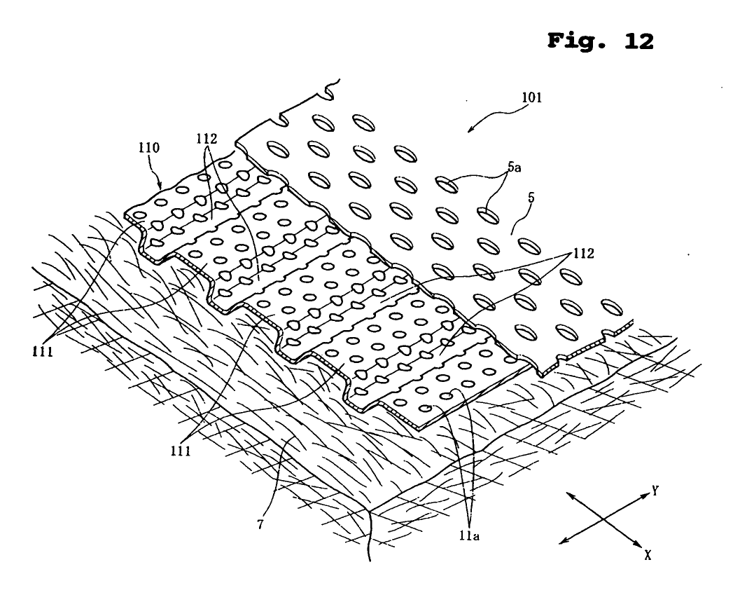

FIG. 11 is an enlarged sectional view corresponding to FIG. 3 and showing a sanitary napkin 101 according to a second embodiment of the present invention, and FIG. 12 is a partial enlarged perspective view corresponding to FIG. 4.

The sanitary napkin 101 according to the second embodiment has an apertured sheet 110 between the topsheet 5 and the liquid absorbent layer 7. The apertured sheet 110 is formed of a resin film that is whitened, treated to be hydrophilic and formed with a number of the liquid passage apertures 11a in the same manner as used for the elements of the barrier layer 11 according to the first embodiment. The individual open area of the liquid passage apertures 11a in the apertured sheet 110 is smaller than that of the liquid passage apertures 5a in the topsheet 5, and the percent open area of the apertured sheet 110 due to the liquid passage apertures 11a is also smaller than that of the topsheet 5 due to the liquid passage apertures 5a. As shown in FIG. 12, the apertured sheet 110 is corrugated to have hills and valleys alternating with each other in the X-direction and extending continuously in the Y-direction. At the hills, the apertured sheet 110 has generally flat top portions which are in contact with the topsheet 5 and function as the elements of a barrier layer 111. Between adjacent elements (i.e., generally flat top portions) of the barrier layer 111, there are defined liquid passage regions 112 where the apertured sheet 110 is away from the topsheet 5 and in contact with the liquid absorbent layer 7 at the bottom of the valleys.

In the sanitary napkin 101, menstrual blood after passing through the liquid passage apertures 5a of the topsheet 5 can be introduced into the valleys of the apertured sheet 110. Menstrual blood flowing down the bottom of the valleys in the Y-direction is drawn by the hydrophilic power of the liquid absorbent layer 7 to pass through the liquid passage apertures 11a for absorption and retention by the liquid absorbent layer 7.

Also in this second embodiment, the liquid passage regions 112 function to prevent lateral leakage of menstrual blood. In addition, since the apertured sheet 110 is laid beneath the topsheet 5, menstrual blood absorbed in the liquid absorbent layer 7 is prevented from easily returning to the topsheet 5 and the color of menstrual blood absorbed in the liquid absorbent layer 7 becomes less noticeable from the outside.

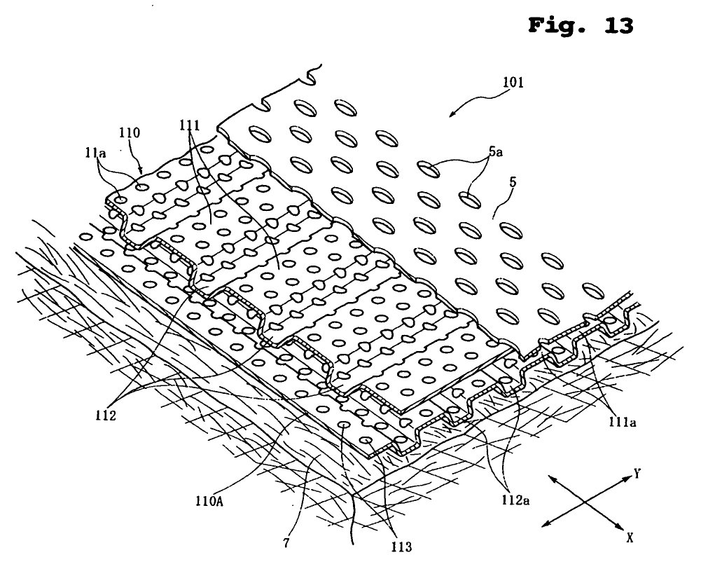

FIG. 13 is a partial enlarged perspective view of a sanitary napkin 101A being a modification of the second embodiment. In the sanitary napkin 101A, another apertured sheet 110A is disposed between the apertured sheet 110 and the liquid absorbent layer 7. The apertured sheet 110A is corrugated to have hills 111a and valleys 112a alternating with each other in the Y-direction and extending continuously in the X-direction. The apertured sheet 110A whose liquid passage apertures are designated 113 may be the same resin film as the overlying apertured sheet 110. However, it is preferred that the individual open area of the liquid passage apertures 113 in the apertured sheet 110A is larger than that of the liquid passage apertures Ha in the apertured sheet 110 and the percent open area of the apertured sheet 110A due to the liquid passage apertures 113 is also larger than that of the apertured sheet 110 due to the liquid passage apertures 11a.

In the sanitary napkin 101A of FIG. 13, menstrual blood after passing through the liquid passage apertures 11a is given to the underlying apertured sheet 110A and then absorbed by the liquid absorbent layer 7 through the liquid passage apertures 113 of the apertured sheet 110A. The apertured sheet 110A also contributes to preventing menstrual blood from returning from the liquid absorbent layer 7 to the topsheet 5 as well as concealing the color of menstrual blood absorbed in the liquid absorbent layer 7.

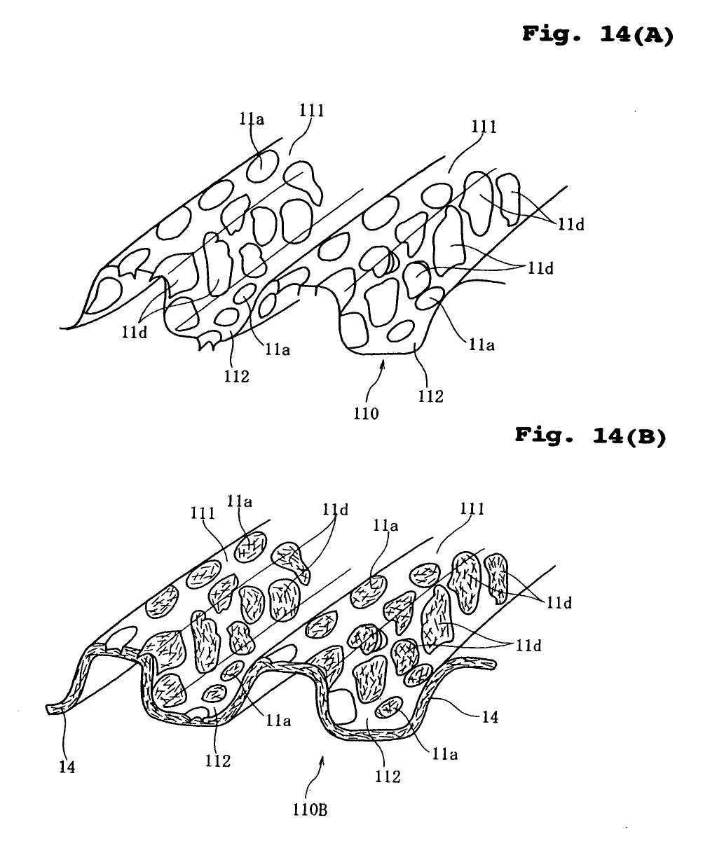

FIG. 14(A) is a partial perspective view showing a preferred structure of the apertured sheet 110. The apertured sheet 110 of FIG. 14(A) is molded into a corrugated configuration such that a resin film formed with liquid passage apertures 11a in advance is held between embossing rolls in meshing engagement with each other. When thus corrugated by embossing, the film, as well as the apertures formed therein, is stretched in the hill slopes (or valley walls) to have apertures 11d. These apertures 11d may have a larger individual open area than the liquid passage apertures 11a left in the top of the hills and the bottom of the valleys.

The apertures 11d facilitate migration of menstrual blood from the liquid passage regions 112 to the liquid absorbent layer 7. Moreover, since the liquid passage apertures 11a left in the top of the hills and the bottom of the valleys are not excessively enlarged, the color of menstrual blood absorbed in the liquid absorbent layer 7 can be effectively concealed.

FIG. 14(B) shows an apertured sheet 110B which is prepared by joining the hydrophilic fibrous layer 14 of FIG. 7 to the garment-side surface of the apertured sheet 110 of FIG. 14(A). Since the hydrophilic fibrous layer 14 is exposed through the apertures 11d, menstrual blood in the liquid passage regions 112 is drawn by the hydrophilic power of the fibrous layer 14 through the apertures 11d to quickly migrate to the liquid absorbent layer 7.

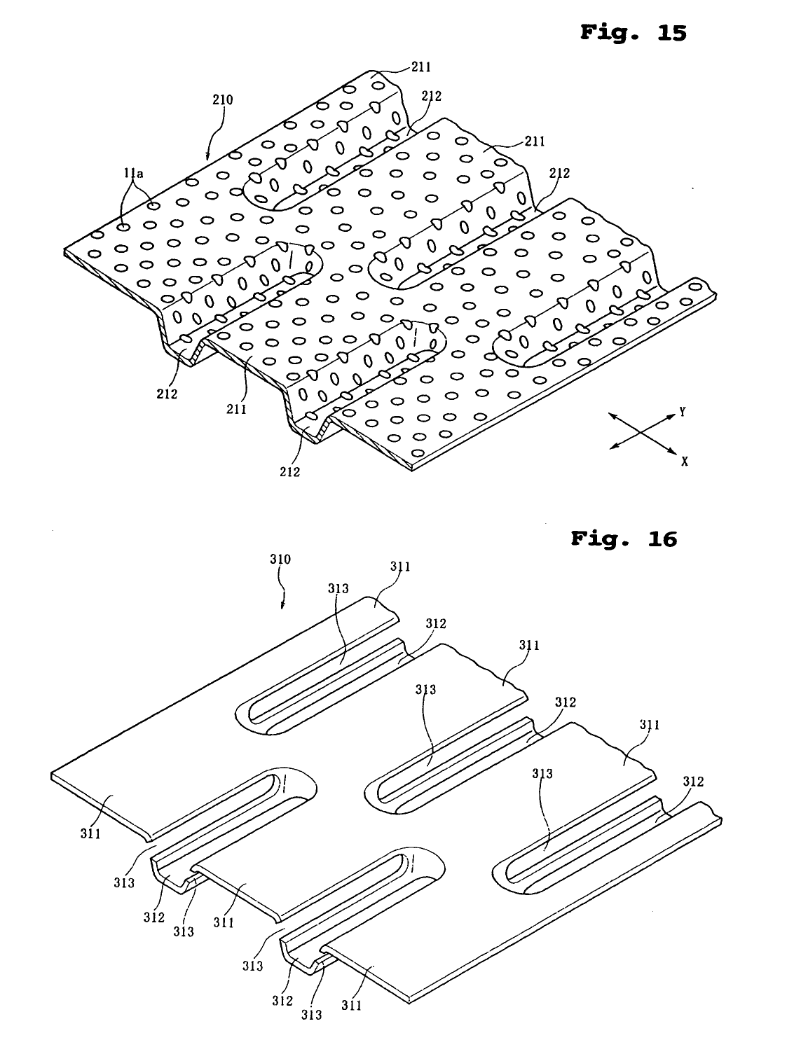

FIGS. 15 to 17 are partial perspective views showing modifications of the apertured sheet disposed beneath the topsheet 5 according to the second embodiment.

FIG. 15 shows an apertured sheet 210 which is formed of a resin film having a large number of the liquid passage apertures 11a like the barrier layer 11 and the apertured sheet 110. The apertured sheet 210 has a plurality of recesses (or hollows) 212 which are elongated in the Y-direction and staggered as shown in FIG. 15. In this apertured sheet 210, the recesses 212 function as the liquid passage regions, while the rest is designated barrier layer 211.

If the apertured sheet 210 of FIG. 15 is molded using embossing rolls in meshing engagement with each other, a plurality of apertures larger than the liquid passage apertures 11a may be formed in the slopes of the recesses 212, as described with reference to FIG. 14(A). It is also possible to join the hydrophilic fibrous layer 14 to the garment-side surface of the apertured sheet 210 and expose the fibrous layer 14 in the slopes of the recesses 212 through the larger apertures, as described with reference to FIG. 14(B).

FIG. 16 shows a sheet 310 which is formed of a resin film whitened and treated to be hydrophilic, but this resin film is not formed with the liquid passage apertures 11a. The sheet 310 has a plurality of recesses (or hollows) 312 which are elongated in the Y-direction and staggered in the same manner as shown in FIG. 15. In laterally opposite slopes of the individual recesses 312, furthermore, apertures (or slots) 313 are formed to extend in the Y-direction. The apertures 313 have a sufficiently larger individual open area than the liquid passage apertures 5a and 11a and have a length equal to or greater than ½ the length of the recesses 312 in the Y-direction.

In this sheet 310, the recesses 312 function as the liquid passage regions, while the rest is designated barrier layer 311. Menstrual blood after passing through the liquid passage apertures 5a of the topsheet 5 is allowed to flow down the recesses 312 in the Y-direction to pass through the apertures 313 for subsequent absorption by the liquid absorbent layer 7. It is, of course, possible to provide the sheet 310 of FIG. 16 with a large number of the liquid passage apertures 11a.

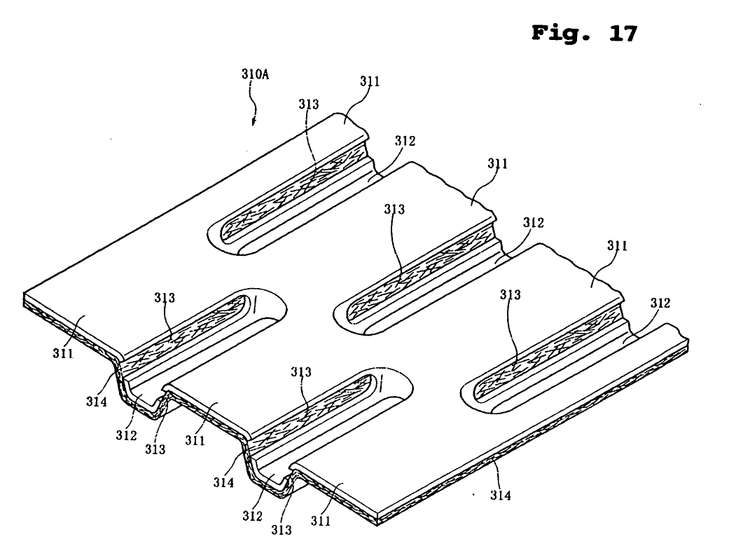

FIG. 17 shows a sheet 310A which is prepared by joining a hydrophilic nonwoven fabric 314 to the garment-side surface of the sheet 310 of FIG. 16. The nonwoven fabric 314 may be a liquid-permeable through-air bonded nonwoven fabric of synthetic resin fibers treated to be hydrophilic. If desired, the nonwoven fabric 314 may contain cellulosic fibers. Since the nonwoven fabric 314 is exposed through the apertures 313, menstrual blood in the recesses 312 is drawn to the nonwoven fabric 314 to migrate to the liquid absorbent layer 7.

It should be noted that the preferred range of the widths of the liquid passage regions 112 in the apertured sheet 110 of FIGS. 11 and 12, the liquid passage regions (recesses 212) of FIG. 15 and the liquid passage regions (recesses 312) of FIGS. 16 and 17 is identical to that of the width Wb shown in FIGS. 3 and 4. Here, the liquid passage regions may have different widths depending on distances from the longitudinal centerline Oy in the X-direction, as described with reference to FIGS. 9 and 10.

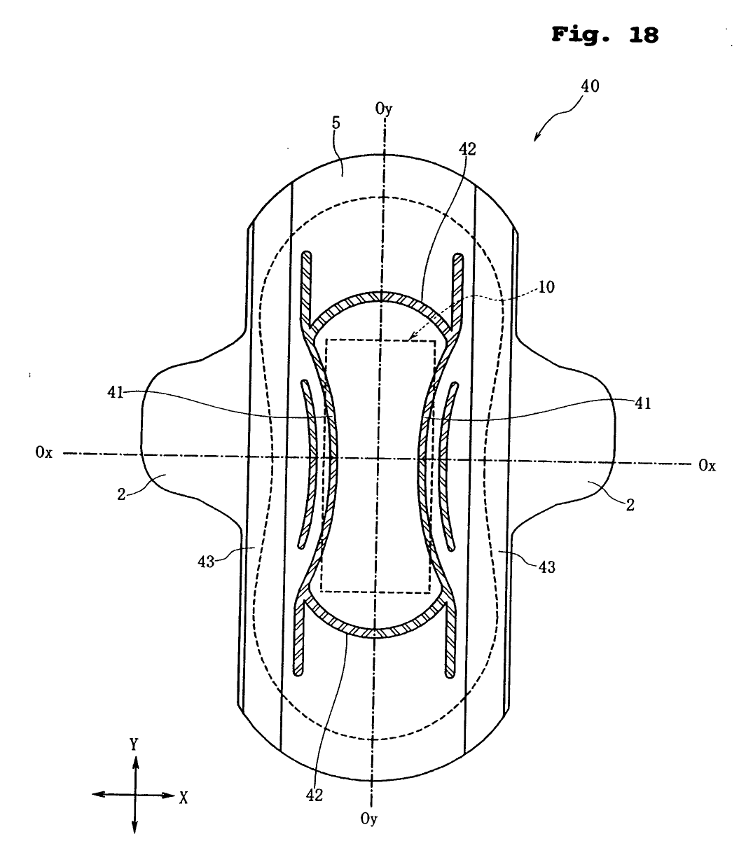

FIG. 18 is a plan view of a sanitary napkin 40 having a different structure according to the present invention. The sanitary napkin 40 further includes: longitudinal compressed grooves 41, 41 overlapping with laterally opposite side portions of the main liquid-receiving region 10 where the barrier layer 11 is present; and lateral compressed grooves 42, 42 spaced forward and rearward from the main liquid-receiving region 10.

### EXAMPLES

The following sanitary napkins were prepared as Examples 1-3 and Comparative Examples 1-3.

### Example 1

Resin film was made of PE (polyethylene) containing 0.3 wt. % of hydrophilizing agent and 7 wt. % of titanium oxide and then processed by a perforation method to have a large number of liquid passage apertures. The basis weight of the resin film was 27 g/m², the individual open area of the liquid passage apertures in the resin film was 0.3 mm², and the percent open area of the resin film due to the liquid passage apertures was 25%. This was used as basic apertured film.

The basic apertured film was cut into strips having a width Wa of 3 mm. These strips were arranged to form the barrier layer 11, as shown in FIGS. 3 and 4. The distance Wb between adjacent strips was 2 mm. That is, the width of the liquid passage regions 12 was 2 mm.

The basic apertured film was stretched to 200% its original length in the X-direction and then used for the topsheet 5. In this stretched apertured film, the liquid passage apertures were elongated in the X-direction.

The liquid absorbent layer 7 was prepared by wrapping fluff pulp having a weight of 500 g/m² in tissue having a basis weight of 15 g/m².

The backsheet 6 was PE film having a basis weight of 23.5 g/m².

Thus, Example 1 was prepared to have the same construction as shown in FIGS. 3 and 4.

### Example 2

The basic apertured film was corrugated as shown in FIGS. 11 and 12. The width of the generally flat top portions of the barrier layer 111 was 3 mm, the width of the liquid passage regions 112 was 2 mm, and the depth of the valleys was 2.0 mm. In order not to close the apertures of the apertured film, a clearance of 0.5 mm was left between the embossing rolls.

The other components were identical to those of Example 1.

### Example 3

As shown in FIG. 6, two barrier layers 11, 11 were stacked one on another such that the elements of the lower barrier layer 11 are located beneath the liquid passage regions 12a defined between the elements of the upper barrier layer 11. For the upper and lower barrier layers 11, 11, the basic apertured film was cut into strips having a width Wa of 3 mm. The width Wb of the liquid passage regions 12a, 12b was 2 mm.

The other components were identical to those of Example 1.

### Comparative Example 1

The basic apertured film was used for the topsheet 5.

Between the topsheet 5 and the liquid absorbent layer 7, there was disposed a through-air bonded nonwoven fabric (basis weight of 20 g/m²; thickness of 0.5 mm) constituted of sheath/core bicomponent synthetic fibers fineness of 4.4 dtex; mean fiber length of 51 mm) of which the core was PP and the sheath was PE.

The other components were identical to those of Example 1.

### Comparative Example 2

The basic apertured film was used for the topsheet 5.

The basic apertured film was also disposed between the topsheet 5 and the liquid absorbent layer 7 without being cut into strips.

The other components were identical to those of Example 1.

### Comparative Example 3

The basic apertured film was stretched to 200% its original length in the X-direction and then used for the topsheet 5 as in Example 1.

The basic apertured film was also disposed between the topsheet 5 and the liquid absorbent layer 7 without being cut into strips.

The other components were identical to those of Example 1.

(Test Method)

The sanitary napkins of Examples 1-3 and Comparative Examples 1-3 were placed on a flat plane with their skin-side surface directed upward and dropping of artificial menstrual blood onto the area where all the layers are stacked one on top of the other (main liquid-receiving region 10) was repeated four times by using an autoburette.

First Drop: 3 g of artificial menstrual blood was applied at a flow rate of 90 g/min.

Second Drop: One minute after completion of the first drop, 4 g of artificial menstrual blood was applied at a flow rate of 96 g/min.

Third Drop: Three minutes after completion of the second drop, 3 g of artificial menstrual blood was applied at a flow rate of 90 g/min.

Fourth Drop: One minute after completion of the third drop, 4 g of artificial menstrual blood was applied at a flow rate of 96 g/min.

Here, the artificial menstrual blood was obtained by adding 300 g of glycerin, 30 g of sodium carboxymethylcellulose and 40 g of sodium chloride to 4 liter of ion-exchanged water, stirring the solution, and coloring it with a food red.

Each time, the time required for the menstrual blood to completely disappear from the skin-side surface of the sample was measured from the beginning of dropping of the artificial menstrual blood.

After completion of the fourth drop, a filter paper, as well as an acrylic plate, was placed on the skin-side surface of the sample, and then, a 5 kg weight was placed on the acrylic plate. After allowed to stand for three minutes, the filter paper, the acrylic plate and the weight were removed and the color difference (ΔE) between white and the color of the surface to which the menstrual blood had been applied was measured using a color-difference meter.

The test results are shown in Table 1.

TABLE 1

| | Com. Ex. 1 | Com. Ex. 2 | Com. Ex. 3 | Ex. 1 | Ex. 2 | Ex. 3 |
|---|---|---|---|---|---|---|
| Penetration Time-1 | 7.7 | 14 | 12 | 7.8 | 7.3 | 9.8 |
| Penetration Time-2 | 8.5 | 15.2 | 14.8 | 11.1 | 6.7 | 8.5 |
| Penetration Time-3 | 10.2 | 17.4 | 9.7 | 7 | 4.4 | 5.5 |
| Penetration Time-4 | 12.8 | 19.4 | 10.9 | 8 | 5.2 | 7.7 |
| Color Difference (ΔE) | 32.4 | 32.3 | 26.8 | 27.9 | 27.3 | 22.5 |

As seen from Table 1, Examples 1-3 are all able to absorb artificial menstrual blood in a short period of time and hardly cause return of artificial menstrual blood from the liquid absorbent layer 7 to the topsheet 5.

Although the present invention has been illustrated and described with respect to exemplary embodiments thereof, it should be understood by those skilled in the art that the foregoing and various other changes, omission and additions may be made therein and thereto, without departing from the spirit and scope of the present invention. Therefore, the present invention should not be understood as limited to the specific embodiments set out above but to include all possible embodiments which can be embodied within a scope encompassed and equivalent thereof with respect to the feature set out in the appended claims.

1. A sanitary napkin comprising:
a liquid-permeable topsheet for facing the vaginal opening of a wearer;
a backsheet located on the opposite side of the topsheet;
a liquid absorbent layer located between the topsheet and the backsheet;
a barrier layer having a plurality of elements and located between the topsheet and the liquid absorbent layer, the elements being spaced laterally and elongated longitudinally of the sanitary napkin, at least a skin-side surface of each element being made of resin;
a plurality of longitudinally elongated liquid passage regions being defined between adjacent elements of the barrier layer to allow liquid passage therethrough and subsequent liquid absorption by the liquid absorbent layer.

2. A sanitary napkin according to claim 1, wherein the individual elements of the barrier layer have a plurality of liquid passage apertures.

3. A sanitary napkin according to claim 2, wherein the topsheet has a skin-side surface made of resin and a plurality of liquid passage apertures, wherein in an area where the topsheet overlaps with the barrier layer, the liquid passage apertures in the individual elements of the barrier layer have a smaller individual open area than the liquid passage apertures in the topsheet and the individual elements of the barrier layer have a smaller percent open area due to the liquid passage apertures than the topsheet.

4. A sanitary napkin according to claim 1, wherein the individual elements of the barrier layer are in contact with the topsheet and the liquid absorbent layer.

5. A sanitary napkin according to claim 1, wherein the barrier layer is of a two-layer structure where an upper barrier layer is located on a lower barrier layer.

6. A sanitary napkin according to claim 5, wherein the upper and lower barrier layers are located in such a staggered manner that the elements of the lower barrier layer are located beneath the liquid passage regions defined between adjacent elements of the upper barrier layer.

7. A sanitary napkin according to claim 1, wherein a corrugated sheet is located between the topsheet and the liquid absorbent layer to function as the barrier layer at top portions thereof.

8. A sanitary napkin according to claim 7, wherein the corrugated sheet is apertured for liquid passage.
